SEQUENCE LISTING (12 sequences)

SEQ ID NO: 1 - DNA, 783 bp, Santalum album
  source 1..783: mol_type = genomic DNA
atgtctgcaatggtttcttctctgactcaagttattggaacctcctcatcccatactgaaaacgtcactccttgtaataagccattggcgactcatgcccaccatcctcatcatcatcagatggtgcaatcatcgacacctgattctgcccccaccccgcaaccgcctctgcaagatcaagaaggatatgcaaggaggcggcactacagaggagtgaggcaaaggccatgggggaaatgggcggccgaaattcgggacccgaagaaaggggcccgcgtgtggctgggaacgttcgagacggccgaggatgctgcagttgcttacgacgaagcggctctgaggttcaaaggcaccaaagctaagctcaacttccccgaaagagtacaaggaaacactcactccattggttacgtggctccccctattaatactacgatgaacaccatggctcctcctcccaccagcaccatgattcttccgcctcgaccgatgccgttagcgtctcaattggacccccaccttctccaatatgcacgccttctttctagtaattgcggagatgatgccaatattccccacattgcttccaccctcttcaatgctcctactccttacggtcatcatcaaggaatgcagtcttcgttggcgactatatcgtcatcatcttcttcttctatgatcatgagtttatcatcaccaacgcagcaacaactttcaatatcatcatcctccacgaactcgagttcctctacttctcatctctgggagaacactgagaattag

SEQ ID NO: 2 - protein, 260 aa, Santalum album
MSAMVSSLTQVIGTSSSHTENVTPCNKPLATHAHHPHHHQMVQSSTPDSAPTPQPPLQDQEGYARRRHYRGVRQRPWGKWAAEIRDPKKGARVWLGTFETAEDAAVAYDEAALRFKGTKAKLNFPERVQGNTHSIGYVAPPINTTMNTMAPPPTSTMILPPRPMPLASQLDPHLLQYARLLSSNCGDDANIPHIASTLFNAPTPYGHHQGMQSSLATISSSSSSSMIMSLSSPTQQQLSISSSSTNSSSSTSHLWENTEN

SEQ ID NO: 3 - DNA, 25 bp, synthetic construct
  source 1..25: mol_type = other DNA
atgtctgcaatggtttcttctctga

SEQ ID NO: 4 - DNA, 23 bp, synthetic construct
  source 1..23: mol_type = other DNA
attctcagtgttctcccagagat

SEQ ID NO: 5 - DNA, 20 bp, synthetic construct
  source 1..20: mol_type = other DNA
agctcaacttccccgaaaga

SEQ ID NO: 6 - DNA, 20 bp, synthetic construct
  source 1..20: mol_type = other DNA
cggaagaatcatggtgctgg

SEQ ID NO: 7 - DNA, 20 bp, synthetic construct
  source 1..20: mol_type = other DNA
ttcatgcgacatctctccga

SEQ ID NO: 8 - DNA, 20 bp, synthetic construct
  source 1..20: mol_type = other DNA
cagcagcccttttacgttgt

SEQ ID NO: 9 - DNA, 20 bp, synthetic construct
  source 1..20: mol_type = other DNA
ttatggtgatcgggcgaagt

SEQ ID NO: 10 - DNA, 20 bp, synthetic construct
  source 1..20: mol_type = other DNA
tatgcgccgagttaatccct

SEQ ID NO: 11 - DNA, 18 bp, synthetic construct
  source 1..18: mol_type = other DNA
tgctcttccccatgccat

SEQ ID NO: 12 - DNA, 20 bp, synthetic construct
  source 1..20: mol_type = other DNA
aacaatttcccgctcagcag

(19) United States
(12) Patent Application Publication
(10) Pub. No.: US 2026/0193667 A1
(43) Pub. Date: Jul. 9, 2026

(54) SANTALUM ALBUM SAERF33 PROTEIN AND/OR SAERF33 GENE, AND APPLICATION OF SAME IN PROMOTING SYNTHESIS OF SANTALUM ALBUM ESSENTIAL OIL

(71) Applicant: The Research Institute of Tropical Forestry, Chinese Academy of Forestry, Guangzhou City (CN)

(72) Inventors: Sen Meng, Guangzhou City (CN); Junkun Lu, Guangzhou City (CN); Fangcuo Qin, Guangzhou City (CN); Yunqing Zhou, Guangzhou City (CN); Yangyan Zhou, Guangzhou City (CN)

(73) Assignee: The Research Institute of Tropical Forestry, Chinese Academy of Forestry, Guangzhou City (CN)

(21) Appl. No.: 19/365,289

(22) Filed: Oct. 22, 2025

(30) Foreign Application Priority Data
Jan. 8, 2025 (CN) 202510027930.4

(51) Int. Cl. C12N 15/82; C07K 14/415

(52) U.S. Cl. CPC C12N 15/8247; C07K 14/415; C12N 15/8205

(57) ABSTRACT

Disclosed in the disclosure are a Santalum album SaERF33 protein and/or an SaERF33 gene, and an application of same in promoting the synthesis of a Santalum album essential oil. An amino acid sequence of the Santalum album SaERF33 protein is as shown in SEQ ID NO.2. In the disclosure, the SaERF33 gene is obtained in Santalum album for the first time, and the overexpression of the SaERF33 gene in Santalum album can significantly promote the synthesis of the Santalum album essential oil, without affecting the normal growth of plants. The disclosure provides a new genetic resource for breeding new Santalum album varieties with high essential oil synthesis efficiency, and has important popularization and application values in breeding Santalum album varieties and producing the Santalum album essential oil.

### REFERENCE TO AN ELECTRONIC SEQUENCE LISTING

The contents of the electronic sequence listing (“USFM25101159_Sequence_Listing.xml”, size is 12,640 bytes and it was created on Dec. 4, 2025) is herein incorporated by reference in its entirety.

### TECHNICAL FIELD

The disclosure relates to the technical field of genetic engineering, and specifically relates to a Santalum album SaERF33 protein and/or an SaERF33 gene, and an application of same in promoting the synthesis of a Santalum album essential oil.

### BACKGROUND

Santalum album is a precious wood, which, featuring unique aroma, is extensively applied in religious ceremonies, perfume manufacturing and traditional medicine. Santalum album essential oil is a natural essential oil extracted from Santalum album wood, famous for its pleasant fragrance and multiple health benefits, such as promoting relaxation, relieving stress and improving sleep quality. In addition, having a certain anti-bacterial and anti-inflammatory function, Santalum album essential oil is widely applied in aromatherapy and skin care products. However, Santalum album is long in growth cycle and low in oil yield, and the high-quality Santalum album essential oil commercially available is in short supply and expensive, limiting the further development and application of the Santalum album essential oil.

The main components of the Santalum album essential oil include santalene and santalol, which invest Santalum album with symbolic fragrance, and play a key role in various bioactivities of the Santalum album essential oil. The santalene includes α-santalene, β-santalene and epi-β-santalene. The santalol includes α-santalol and β-santalol. The santalene is converted from farnesyl diphosphate (FPP) by means of santalene synthetase SaSSY (a member of the terpene synthase (TPS) family), and the santalene is further catalyzed into santalol by means of P450 oxidase SaCYP736A167. It is a new challenge for those skilled in the field to obtain new Santalum album varieties with higher essential oil synthesis efficiency and improve the yield of the Santalum album essential oil efficiently and stably by means of advanced genetic engineering technique.

### SUMMARY

To solve the above problems existing in the prior art, the disclosure provides a Santalum album SaERF33 protein and/or an SaERF33 gene, and an application of same in promoting the synthesis of a Santalum album essential oil.

A first objective of the disclosure is to provide a Santalum album SaERF33 protein.

A second objective of the disclosure is to provide an application of the Santalum album SaERF33 protein and/or an SaERF33 gene in promoting the synthesis of a Santalum album essential oil.

A third objective of the disclosure is to provide an application of the Santalum album SaERF33 protein and/or the SaERF33 gene in promoting the synthesis of a santalene and/or a santalol.

A fourth objective of the disclosure is to provide an application of a reagent promoting the expression of the Santalum album SaERF33 protein and/or the SaERF33 gene in promoting the synthesis of the Santalum album essential oil.

A fifth objective of the disclosure is to provide an application of the reagent promoting the expression of the Santalum album SaERF33 protein and/or the SaERF33 gene in promoting the synthesis of the santalene and/or the santalol.

A sixth objective of the disclosure is to provide a biomaterial for promoting the synthesis of the Santalum album essential oil.

A seventh objective of the disclosure is to provide an application of the biomaterial in promoting the synthesis of the Santalum album essential oil.

An eighth objective of the disclosure is to provide a product for promoting the synthesis of the Santalum album essential oil.

A ninth objective of the disclosure is to provide a method for promoting the synthesis of the Santalum album essential oil.

To realize the above objectives, the disclosure employs the following technical solutions.

In the disclosure, an SaERF33 gene is discovered in Santalum album, and the SaERF33 gene encodes an SaERF33 protein, and the important function of the SaERF33 gene in regulating the synthesis of a Santalum album essential oil is clarified by means of tissue culture and genetic engineering technique.

The disclosure sets forth the following content.

A Santalum album SaERF33 protein is provided, which has an amino acid sequence as shown in SEQ ID NO.2.

An application of the Santalum album SaERF33 protein and/or an SaERF33 gene in promoting the synthesis of a Santalum album essential oil is provided, with the amino acid sequence of the Santalum album SaERF33 protein being as shown in SEQ ID NO.2.

Preferably, the Santalum album essential oil is a Santalum album essential oil from Indian Santalum album.

An application of the Santalum album SaERF33 protein and/or the SaERF33 gene in promoting the synthesis of a santalene and/or a santalol is provided, with the amino acid sequence of the Santalum album SaERF33 protein being as shown in SEQ ID NO.2.

Preferably, the santalene and/or the santalol is a santalene and/or a santalol from Indian Santalum album.

Preferably, the santalene includes one or more of α-santalene, β-santalene and epi-β-santalene.

Preferably, the santalol includes α-santalol.

An application of a reagent promoting the expression of the Santalum album SaERF33 protein and/or the SaERF33 gene in promoting the synthesis of the Santalum album essential oil is provided, with the amino acid sequence of the Santalum album SaERF33 protein being as shown in SEQ ID NO.2.

Preferably, the Santalum album essential oil is the Santalum album essential oil from Indian Santalum album.

An application of the reagent promoting the expression of the Santalum album SaERF33 protein and/or the SaERF33 gene in promoting the synthesis of the santalene and/or the santalol is provided, with the amino acid sequence of the Santalum album SaERF33 protein being as shown in SEQ ID NO.2.

Preferably, the santalene and/or the santalol is the santalene and/or the santalol from Indian Santalum album.

Preferably, the santalene includes one or more of α-santalene, β-santalene and epi-β-santalene.

Preferably, the santalol includes α-santalol.

A biomaterial for promoting the synthesis of the Santalum album essential oil is provided, which is any one of the following (1) to (4):
(1) a nucleic acid molecule encoding the Santalum album SaERF33 protein, the amino acid sequence of the Santalum album SaERF33 protein being as shown in SEQ ID NO.2;
(2) an expression cassette containing the nucleic acid molecule in (1);
(3) a recombinant expression vector containing the nucleic acid molecule in (1); and
(4) a microorganism containing the recombinant expression vector in (3).

Preferably, a nucleotide sequence of the nucleic acid molecule in (1) is as shown in SEQ ID NO.1 or as shown in a complete complementary sequence of the sequence as shown in SEQ ID NO:1.

Preferably, the recombinant expression vector in (3) takes a pBWA(V)HS-GFP vector as a skeleton.

More preferably, the recombinant expression vector in (3) takes the pBWA(V)HS-GFP vector as the skeleton, and the nucleic acid molecule in (1) is arranged between Smal site and XbaI site.

In the disclosure, there are no specific limits on the source and type of the microorganism in (4). The conventional microorganisms, including but not limited to Escherichia coli, Agrobacterium, etc., capable of carrying the recombinant expression vector can achieve the objectives of the disclosure.

Preferably, the microorganism in (4) is Agrobacterium.

More preferably, the Agrobacterium is Agrobacterium tumefaciens.

Further preferably, the Agrobacterium tumefaciens is an LBA4404 strain.

An application of any one of the biomaterials in promoting the synthesis of the Santalum album essential oil is provided.

Preferably, the Santalum album essential oil is the Santalum album essential oil from Indian Santalum album.

An application of any one of the biomaterials in promoting the synthesis of the santalene and/or the santalol.

Preferably, the Santalum album essential oil is the santalene and/or the santalol from Indian Santalum album.

Preferably, the santalene includes one or more of α-santalene, β-santalene and epi-β-santalene.

Preferably, the santalol includes α-santalol.

A product for promoting the synthesis of the Santalum album essential oil is provided, including the biomaterial for promoting the synthesis of the Santalum album essential oil. The biomaterial is any one of the following (1) to (4):
(1) a nucleic acid molecule encoding the Santalum album SaERF33 protein, the amino acid sequence of the Santalum album SaERF33 protein being as shown in SEQ ID NO.2;
(2) an expression cassette containing the nucleic acid molecule in (1);
(3) a recombinant expression vector containing the nucleic acid molecule in (1); and
(4) a microorganism containing the recombinant expression vector in (3).

Preferably, a nucleotide sequence of the nucleic acid molecule in (1) is as shown in SEQ ID NO.1 or as shown in a complete complementary sequence of the sequence as shown in SEQ ID NO:1.

Preferably, the recombinant expression vector in (3) takes a pBWA(V)HS-GFP vector as a skeleton.

More preferably, the recombinant expression vector in (3) takes the pBWA(V)HS-GFP vector as the skeleton, and the nucleic acid molecule in (1) is arranged between Smal site and XbaI site.

In the disclosure, there are no specific limits on the source and type of the microorganism in (4). The conventional microorganisms, including but not limited to Escherichia coli, Agrobacterium, etc., capable of carrying the recombinant expression vector can achieve the objectives of the disclosure.

Preferably, the microorganism in (4) is Agrobacterium.

More preferably, the Agrobacterium is Agrobacterium tumefaciens.

Further preferably, the Agrobacterium tumefaciens is an LBA4404 strain.

An application of any one of the products in promoting the synthesis of the Santalum album essential oil is provided.

Preferably, the Santalum album essential oil is the Santalum album essential oil from Indian Santalum album.

An application of any one of the products in promoting the synthesis of the santalene and/or the santalol is provided.

Preferably, the Santalum album essential oil is the santalene and/or the santalol from Indian Santalum album.

Preferably, the santalene includes one or more of α-santalene, β-santalene and epi-β-santalene.

Preferably, the santalol includes α-santalol.

A method for promoting the synthesis of the Santalum album essential oil includes improving an expression of the Santalum album SaERF33 protein and/or the SaERF33 gene in Santalum album.

Preferably, any one of the biomaterials or any one of the products is used to treat Santalum album, to improve the expression of the Santalum album SaERF33 protein and/or the SaERF33 gene in Santalum album.

Preferably, the promoting the synthesis of the Santalum album essential oil includes promoting the synthesis of the santalene and/or the santalol.

More preferably, the santalene includes one or more of α-santalene, β-santalene and epi-β-santalene.

More preferably, the santalol includes α-santalol.

Preferably, Santalum album is Indian Santalum album.

Compared with the prior art, the disclosure has the following beneficial effects.

In the disclosure, the SaERF33 gene is obtained in Santalum album for the first time, and the overexpression of the SaERF33 gene in Santalum album can significantly promote the synthesis of the Santalum album essential oil, without affecting the normal growth of plants. The disclosure provides a new genetic resource for breeding new Santalum album varieties with high essential oil synthesis efficiency, and has important popularization and application values in breeding Santalum album varieties and producing the Santalum album essential oil.

### BRIEF DESCRIPTION OF THE DRAWINGS

FIG. 1 shows electrophoretic detection results of polymerase chain reaction (PCR) amplification of a coding DNA sequence (CDS) region (SEQ ID NO.1) of an SaERF33 gene, where M represents DNA Markers (from small to large, they are 100 bp, 200 bp, 500 bp, 750 bp, 1000 bp, 1500 bp, 2000 bp, 3000 bp and 5000 bp).

FIG. 2 shows the electrophoretic detection results of PCR of a monoclonal bacteria solution, with 1 to 3 representing 3 monoclones, and M representing DNA Markers (from small to large, they are 100 bp, 200 bp, 500 bp, 750 bp, 1000 bp, 1500 bp, 2000 bp, 3000 bp and 5000 bp).

FIG. 3 shows a map of a Santalum album SaERF33 overexpression vector.

FIG. 4 shows photographs of transformation of positive callus of LBA4404-SaERF33 Agrobacterium, taken under fluorescent and bright field conditions.

FIG. 5 shows photographs of wild-type (WT) plants and overexpression (OE) plants, with #16, #37, #41, and #42 all being OE plants.

FIG. 6 displays the statistical results of the mRNA relative expression of SaERF33 in transgenic plants, where WT represents WT plants, and different lowercase letters indicate significant differences between groups (p<0.05), and the same lowercase letters indicate non-significant differences between groups (p≥0.05).

FIG. 7 shows the statistical results of the mRNA relative expression of SaSSY and SaCYP736A167 in transgenic plants, where WT represents WT plants, and different lowercase letters indicate significant differences between groups (p<0.05), and the same lowercase letters indicate non-significant differences between groups (p≥0.05).

FIG. 8A represents the content of α-santalene;

FIG. 8B represents the content of β-santalene;

FIG. 8C represents the content of epi-β-santalene; and

FIG. 8D represents the content of α-santalol, where WT represents WT plants, and different lowercase letters indicate significant differences between groups (p<0.05), and the same lowercase letters indicate non-significant differences between groups (p≥0.05).

### DETAILED DESCRIPTION

The disclosure is further described in combination with the accompanying drawings of the specification and specific embodiments. The embodiments only serve for explaining the disclosure, rather than limiting the scope of the disclosure. The experimental methods used in the embodiments mentioned below are conventional unless otherwise specified. The materials, reagents, etc. used are commercially available unless otherwise specified.

### Embodiment 1 Construction of Santalum album SaERF33 Overexpression Vector

### 1. Target Fragment Amplification

On the basis of genomic sequencing data of Santalum album (variety: Indian Santalum album), it was found that an SaERF33 gene has a CDS region with the sequence as:

(SEQ ID NO. 1)
ATGTCTGCAATGGTTTCTTCTCTGACTCAAGTTATTGGAACCTCCTCATC
CCATACTGAAAACGTCACTCCTTGTAATAAGCCATTGGCGACTCATGCCC
ACCATCCTCATCATCATCAGATGGTGCAATCATCGACACCTGATTCTGCC
CCCACCCCGCAACCGCCTCTGCAAGATCAAGAAGGATATGCAAGGAGGCG
GCACTACAGAGGAGTGAGGCAAAGGCCATGGGGGAAATGGGCGGCCGAAA
TTCGGGACCCGAAGAAAGGGGCCCGCGTGTGGCTGGGAACGTTCGAGACG
GCCGAGGATGCTGCAGTTGCTTACGACGAAGCGGCTCTGAGGTTCAAAGG
CACCAAAGCTAAGCTCAACTTCCCCGAAAGAGTACAAGGAAACACTCACT
CCATTGGTTACGTGGCTCCCCCTATTAATACTACGATGAACACCATGGCT
CCTCCTCCCACCAGCACCATGATTCTTCCGCCTCGACCGATGCCGTTAGC
GTCTCAATTGGACCCCCACCTTCTCCAATATGCACGCCTTCTTTCTAGTA
ATTGCGGAGATGATGCCAATATTCCCCACATTGCTTCCACCCTCTTCAAT
GCTCCTACTCCTTACGGTCATCATCAAGGAATGCAGTCTTCGTTGGCGAC
TATATCGTCATCATCTTCTTCTTCTATGATCATGAGTTTATCATCACCAA
CGCAGCAACAACTTTCAATATCATCATCCTCCACGAACTCGAGTTCCTCT
ACTTCTCATCTCTGGGAGAACACTGAGAATTAG,

the gene encodes the SaERF33 protein, and the amino acid sequence from terminal N to terminal C is: terminal

(SEQ ID NO. 2)
N-MSAMVSSLTQVIGTSSSHTENVTPCNKPLATHAHHPHHHQMVQSSTPD
SAPTPQPPLQDQEGYARRRHYRGVRQRPWGKWAAEIRDPKKGARVWLGTF
ETAEDAAVAYDEAALRFKGTKAKLNFPERVQGNTHSIGYVAPPINTTMNT
MAPPPTSTMILPPRPMPLASQLDPHLLQYARLLSSNCGDDANIPHIASTL
FNAPTPYGHHQGMQSSLATISSSSSSSMIMSLSSPTQQQLSISSSSTNSS
SSTSHLWENTEN*.

Using the genomic cDNA from root tissues of Santalum album (variety: Indian Santalum album) as a template, the upstream cloning primer SaERF33-F (5′-ATGTCTGCAATGGTTTCTTCTCTGA-3′ (SEQ ID NO.3)) and the downstream cloning primer SaERF33-R (5′-ATTCTCAGTGTTCTCCCAGAGAT-3′ (SEQ ID NO.4)) are utilized to perform PCR amplification. A gene cloning kit (Vazyme) was utilized for the PCR amplification. A PCR reaction system included: 10 μL of 2×Phanta Max Master Mix; 8 μL of ddH₂O; 0.5 μL of SaERF33-F (SEQ ID NO.3); 0.5 μL of SaERF33-R (SEQ ID NO.4); and 1 μL of cDNA.

PCR amplification products were collected and subjected to agarose gel electrophoresis detection. The electrophoretic results are as shown in FIG. 1, it can be seen that the bands of the PCR amplification products are single and the size thereof is consistent with the length of the CDS region (SEQ ID NO.1) of the SaERF33 gene.

### 2. Vector Ligation

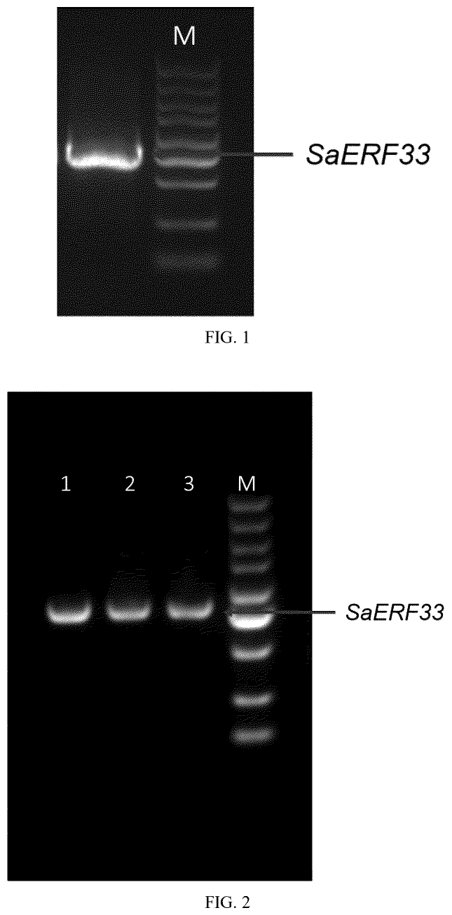

Using double digests and homologous recombination methods, restriction enzymes Smal and XbaI were employed for ligating the CDS region (SEQ ID NO.1) of the SaERF33 gene amplified in the previous step into a pBWA(V)HS-GFP vector (which expresses green fluorescence). Subsequently, a ligation product was transformed into Escherichia coli, followed by spreading a converted product onto an LB solid medium. A monoclone was selected and subjected to expansion culture. A bacteria solution was collected for PCR identification. As shown in FIG. 2, each monoclone has a single band of the expected size, which is the positive monoclone.

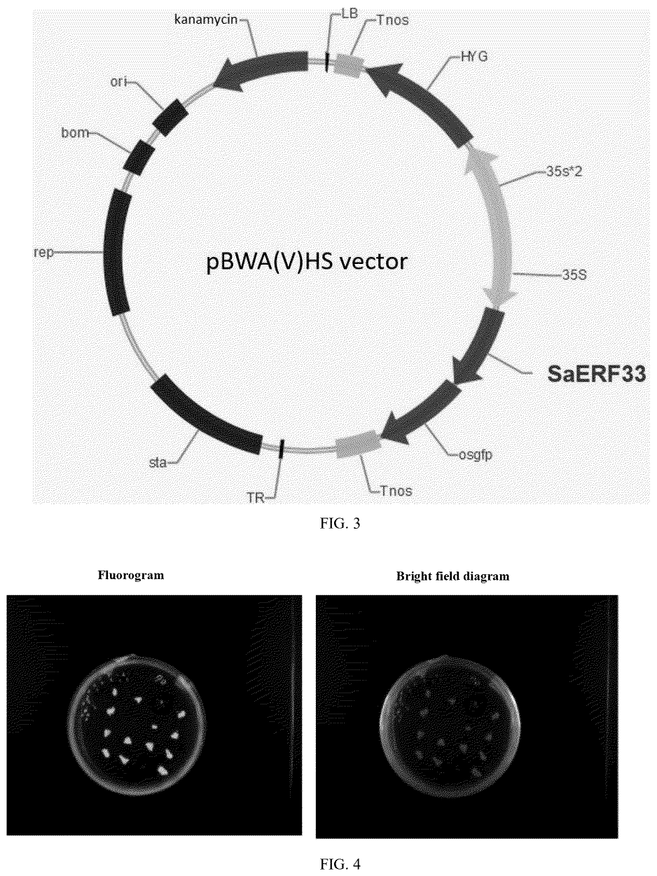

Plasmids were extracted from the positive monoclone and subjected to sequencing identification. The plasmids with correct sequences shown in sequencing results were defined as pBWA(V)HS-SaERF33 vectors, indicating the successful construction of the Santalum album SaERF33 overexpression vectors as shown in FIG. 3.

### Embodiment 2 Construction of Santalum album SaERF33 Overexpression Strain Line

### 1. Construction of Agrobacterium

The pBWA(V)HS-SaERF33 vector obtained from Embodiment 1 was introduced into Agrobacterium tumefaciens (LBA4404 strain) using the freeze-thaw method, and Agrobacterium containing the Santalum album SaERF33 overexpression vector was obtained, which was defined as LBA4404-SaERF33 Agrobacterium.

### 2. Culture and Transformation of Callus

Young stem segments, approximately 1.0 centimeter in length, were selected from explants (preserved at the Research Institute of Tropical Forestry, Chinese Academy of Forestry, and available to the public upon request from the applicant) of Santalum album (variety: Indian Santalum album), and were inoculated onto a callus induction medium (a Murashig and Skoog (MS) medium containing 1.0 mg/L of 2,4-dichlorophenoxyacetic acid (2,4-D), with a pH of 5.8), and subsequently subcultured until they were 4 weeks of age.

The 4-week-old Santalum album callus was co-cultured with LBA4404-SaERF33 Agrobacterium for 20 minutes and then transferred to an induction medium added with callus for continued culture. After 1 week, the callus was transferred to a selection medium (an MS medium containing 40 mg/L of hygromycin, with a pH of 5.8) for screening. Under excitation light, wild-type callus did not exhibit green fluorescence. As shown in FIG. 4, under excitation light, the callus containing the pBWA(V)HS-SaERF33 vector expresses green fluorescence, indicating that the positive callus of LBA4404-SaERF33 Agrobacterium is successfully transformed, with this callus being defined as OE callus.

### 3. Culture and Identification of Transgenic Plants

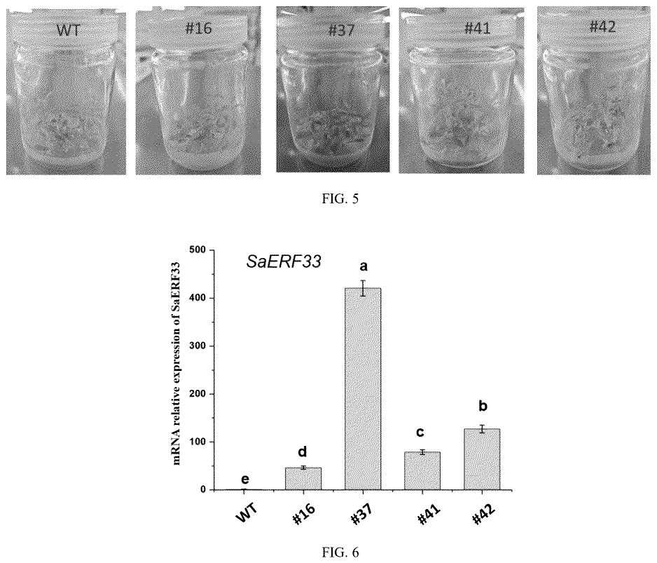

OE callus and WT callus of the same age were separately transferred to a shoot induction medium (an MS medium containing 0.1 mg/L of 6-benzylaminopurine (6-BA) and 0.1 mg/L of indole-3-butyric acid (IBA), with a pH of 5.8) and cultured for 2 months. Subsequently, they were separately transferred to a rooting medium (an MS medium containing 0.2 mg/L of IBA and 0.2 mg/L of naphthaleneacetic acid (NAA), with a pH of 5.8) for continued culture. The transgenic plants obtained from the culture of OE callus were defined as OE plants, and the plants obtained from the culture of WT callus were defined as WT plants. After 1 month, the growth status of the OE plants (#16, #37, #41, and #42) and WT plants is shown in FIG. 5, with no significant differences in appearance and growth vigor.

The cultured root tissues were separately collected from the OE plants and WT plants, and RNA was extracted and subjected to reverse transcription to obtain cDNA. Using the cDNA as a template, the mRNA expression of relevant genes was detected by utilizing the primers listed in Table 1 and fluorescent quantitative PCR technique. A fluorescent quantitative PCR reaction system included: 10 μL of 2×SYBR Green qPCR Mix; 8 μL of RNase free delH₂O; 1 μL of cDNA; 0.5 μL of upstream quantitative primer (F); and 0.5 μL of downstream quantitative primer (R).

TABLE 1
Fluorescent quantitative PCR primers

| Gene | Primer sequence (5′→3′) |
|---|---|
| SaERF33 | F: AGCTCAACTTCCCCGAAAGA |
| | (SEQ ID NO. 5) |
| | R: CGGAAGAATCATGGTGCTGG |
| | (SEQ ID NO. 6) |
| SaSSY | F: TTCATGCGACATCTCTCCGA |
| | (SEQ ID NO. 7) |
| | R: CAGCAGCCCTTTTACGTTGT |
| | (SEQ ID NO. 8) |
| SaCYP736A167 | F: TTATGGTGATCGGGCGAAGT |
| | (SEQ ID NO. 9) |
| | R: TATGCGCCGAGTTAATCCCT |
| | (SEQ ID NO. 10) |
| Actin | F: TGCTCTTCCCCATGCCAT |
| (internal | (SEQ ID NO. 11) |
| reference) | R: AACAATTTCCCGCTCAGCAG |
| | (SEQ ID NO. 12) |

As shown in FIG. 6, compared with WT plants, the transcriptional levels of SaERF33 in the four OE plants (#16, #37, #41, and #42) are significantly increased, with #37 exhibiting the highest transcriptional level of SaERF33, indicating that the Santalum album SaERF33 overexpression strain line is successfully constructed.

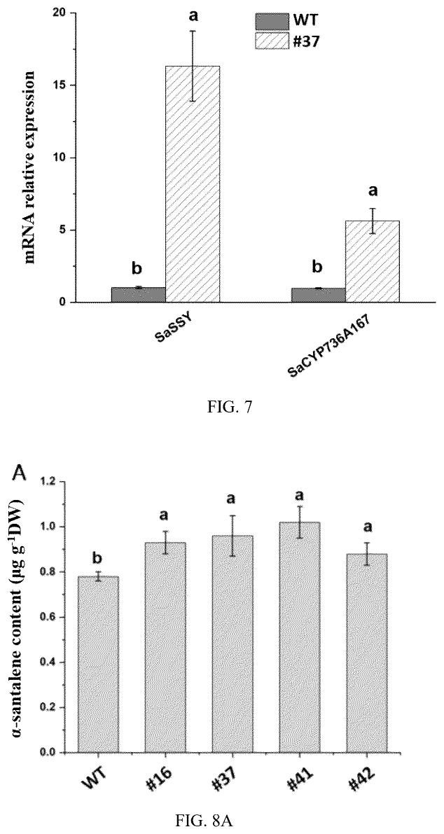

As shown in FIG. 7, compared with WT plants, the transcriptional levels of SaSSY and SaCYP736A167 in #37 are significantly increased. Similar detection results are also observed in the other three OE plants (#16, #41, and #42), indicating that overexpressing the SaERF33 gene in Santalum album can upregulate the terpene synthesis pathway related to the Santalum album essential oil.

### Embodiment 3 Synthesis of Santalum album Essential Oil of Santalum album SaERF33 Overexpression Strain Line

1. Determination of Santalum album Essential Oil

Root tissues were collected from WT plants and four OE plants (#16, #37, #41 and #42) in Embodiment 2, and were detected according to the following method: 0.1 g of Santalum album roots were ground and dried, and 1 mL of hexane was used for extracting metabolite by repeatedly shaking upside down, lasting for 3 days. An extraction solution was centrifuged at 2000 rpm/min for 5 minutes, and a supernatant was extracted and blown to dry with nitrogen and evaporated until dried. The dried residue was redissolved in 50 μL of hexane, and dodecane (0.1 mg/mL) was added as an internal standard. The detection was performed according to the following method: a GC-2010 gas chromatograph (Shimadzu, Suzhou, China) system was operated in the electron ionization selected ion monitoring mode. Santalum album essential oil samples were analyzed on a DB Wax fused silica column (Agilent Technologies) (30 m in length, 250 μm in inner diameter, and 0.25 μm in film thickness). A sample injector was operated in the pulsed splitless mode, with a temperature of the sample injector maintained at 250° C. Helium was used as a carrier gas at a flow rate of 0.8 mL/min, and a pulse pressure was set to 25 psi, lasting for 0.5 minutes. The scan range was: m/z 40-500; and SIM: m/z 93, 94, 105, 107, 119, 122, and 202. The dwell time was 50 milliseconds. The oven program included: 50° C. for 3 minutes; heating at 5° C./min to 250° C. and holding for 2 minutes; and then heating at 5° C./min to 300° C. and holding for 15 minutes. Data acquisition and processing were performed using ChemStation software (Agilent Technologies). Compounds were identified by comparing mass spectra with the NIST/EPA/NIH Mass Spectral Library version 2.0 (http://chemdata.nist.gov/). The relative abundance of various components of essential oil was calculated by manually integrating the peak areas and normalizing using an internal standard and the amount of tissue used (dry weight). The analysis was performed using three biological replicates, and each biological replicate contained two technical replicates.

### 2. Determination Results

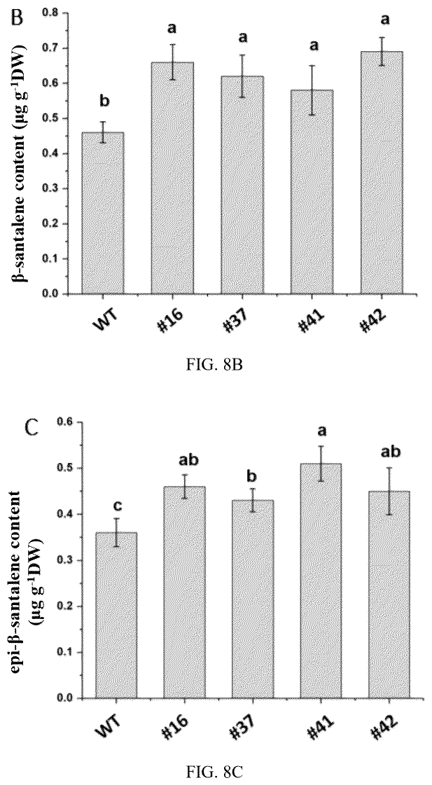

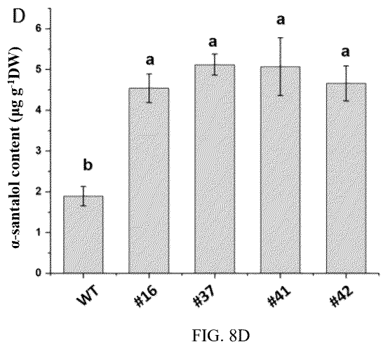

As shown in FIGS. 8A-8D, the contents of α-santalene, β-santalene, epi-β-santalene and α-santalol in the four OE plants (#16, #37, #41 and #42) are significantly increased compared with those in WT plants. It is indicated that the overexpression of the SaERF33 gene in Santalum album can promote the synthesis of the Santalum album essential oil.

Finally, it is to be noted that the above embodiments only serve for illustrating the technical solutions of the disclosure rather than limiting the scope of protection of the disclosure. For those of ordinary skill in the art, other variations or modifications in different forms can be made on the basis of the above descriptions and ideas. It is neither necessary nor possible to list all the implementations herein. Any modifications, equivalents and improvements made within the spirit and principle of the disclosure are included in the scope of claims of the disclosure.

What is claimed is:

1. A Santalum album SaERF33 protein, having an amino acid sequence as shown in SEQ ID NO.2.

2. An application of the Santalum album SaERF33 protein and/or an SaERF33 gene in promoting the synthesis of a Santalum album essential oil, the amino acid sequence of the Santalum album SaERF33 protein being as shown in SEQ ID NO.2.

3. An application of the Santalum album SaERF33 protein and/or the SaERF33 gene in promoting the synthesis of a santalene and/or a santalol, the amino acid sequence of the Santalum album SaERF33 protein being as shown in SEQ ID NO.2.

4. A biomaterial for promoting the synthesis of the Santalum album essential oil, being any one of the following (1) to (4):
(1) a nucleic acid molecule encoding the Santalum album SaERF33 protein according to claim 1;
(2) an expression cassette containing the nucleic acid molecule in (1);
(3) a recombinant expression vector containing the nucleic acid molecule in (1); and
(4) a microorganism containing the recombinant expression vector in (3).

5. The biomaterial according to claim 4, wherein a nucleotide sequence of the nucleic acid molecule in (1) is as shown in SEQ ID NO.1.

6. An application of the biomaterial according to claim 4 in promoting the synthesis of the Santalum album essential oil.

7. A product for promoting the synthesis of the Santalum album essential oil, comprising the biomaterial according to claim 4.

8. A method for promoting the synthesis of the Santalum album essential oil, comprising improving an expression of the Santalum album SaERF33 protein and/or the SaERF33 gene in Santalum album, the amino acid sequence of the Santalum album SaERF33 protein being as shown in SEQ ID NO.2.
